(19) United States
(12) Patent Application Publication
(10) Pub. No.: US 2008/0125382 A1
(43) Pub. Date: May 29, 2008

(54) Extracellular superoxide dismutase (EC-SOD) gene delivery to prevent oxidative injury

(75) Inventors: Jian Wu, Davis, CA (US); Michael H. Nantz, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Regents of the University of California, Oakland, CA (US)

(21) Appl. No.: 11/040,489

(22) Filed: Jan. 20, 2005

Related U.S. Application Data
(60) Provisional application No. 60/537,926, filed on Jan. 20, 2004.

(51) Int. Cl. A61K 31/70; A01N 1/00; A61N 1/30; A61P 43/00; C07H 21/04; C12N 15/87

(52) U.S. Cl. 514/44; 435/1.1; 435/455; 536/22.1; 604/19

(57) ABSTRACT

The present invention relates to compositions and methods for preventing oxidative injury to a cell. The compositions comprise a lipid formulation and a recombinant nucleic acid encoding an extracellular superoxide dismutase (EC-SOD), which upon transfection of a target cell is expressed by the cell and acts as an enzymatic antioxidant. The invention also provides for an isolated organ treated with the claimed composition or method.

### RELATED APPLICATION

This application claims the benefit of U.S. provisional patent application No. 60/537,926, filed Jan. 20, 2004, the contents of which are incorporated herein by reference in the entirety.

### STATEMENT AS TO RIGHTS TO INVENTIONS MADE UNDER FEDERALLY SPONSORED RESEARCH OR DEVELOPMENT

This invention was made with government support under Grant No. DK-09762 of the National Institute of Diabetes and Digestive and Kidney Diseases. The government has certain rights in this invention.

### BACKGROUND OF THE INVENTION

Reactive oxygen species (ROS) are generated during a variety of physiological and pathological processes, e.g., metabolism, aging, and carcinogenesis. Common free radicals, such as superoxide anions (O₂.⁻), hydroxyl radicals (HO.⁻), hydrogen peroxide (H₂O₂), or hydroxyethyl radicals (HER), are generated in the liver, for example, by drug toxicity, ischemia-reperfusion, alcohol metabolism, and reactive intermediate metabolites of hepatotoxins or drugs (Wu et al., Exp. Opin. Invest. Drugs, 8:585-607 (1999)). Oxidative damage due to these ROS is responsible for necrosis and/or apoptosis of many cell types and causes various types of tissue damages. For instance, oxidative stress causes death in hepatocytes and sinusoidal endothelial cells (SEC), and subsequently leads to liver inflammation and hepatic fibrogenesis (Wu et al., J. Gastroenterol., 35:665-672 (2000)).

SOD is an enzyme that mediates the dismutation of superoxide anions and is therefore an enzymatic antioxidant. There are three isozymes of SOD. The copper-zinc-containing form of SOD (CuZn-SOD or SOD₁), which is localized in the cytosol and nucleus of all cell types, plays a major role in the intracellular antioxidative system. Manganese SOD (Mn-SOD or SOD₂) is a manganese-containing enzyme localized in the matrix of mitochondria. The third type is extracellular SOD (EC-SOD, or SOD₃), which is a secretory glycoprotein composed of four 30 kD subunits, each containing a Cu atom and a Zn atom (Marklund, Methods Enzymol. 349:74-80 (2002)). EC-SOD is localized primarily in the interstitial matrix of tissue, and characterized by its high affinity for heparan sulfate binding (Marklund, J. Clin. Invest. 74:1398-1403 (1984)). The importance of EC-SOD under normal and pathologic conditions has not been fully elucidated. It may play a role in regulating superoxide anion levels in the extracellular space since the superoxide anion poorly penetrates the cell membrane when it can be cleared by intracellular CuZn-SOD (Marklund, Methods Enzymol., 349:74-80 (2002); Winterbourn et al., J. Clin. Invest. 80:1486-1491 (1987)). Thus, EC-SOD may be an important factor in the extracellular space to degrade superoxide anions generated during pathologic processes and to protect tissues from ROS toxicity.

ROS that exist in the extracellular space appear to mediate the interactions among different cell types. Therefore, treatments that reduce the production of ROS, inhibit the release of ROS, or inactivate their toxic action should attenuate ROS-associated liver injury. Emerging strategies include the administration of anti-oxidants (Yao et al., Am. J. Physiol., 267:G476-G484 (1994); Ferret et al., Hepatology, 33:1173-1180 (2001); Malassagne et al., Gastroenterology, 121:1451-1459 (2001)) and the gene delivery of free radical scavengers, such as Cu/Zn-SOD by adenoviral vectors (Wheeler et al., Gastroenterology, 120:1241-1250 (2001)). Adenoviral EC-SOD vectors were also used to protect rabbits from myocardial infarction damage (Li et al., Circulation, 103:1893-1898 (2001)) and from ischemia/reperfusion-associated liver injury in mice (Wheeler et al., Human Gene Ther. 12:2167-2177 (2001)). Gene delivery will overcome the short half-life of the enzyme in the body. Adenoviral vectors, however, present some obvious drawbacks such as liver toxicity, immunogenicity of viral products, etc.

There exists the need to develop new and improved anti-oxidative compositions and methods, such as those using EC-SOD to provide anti-oxidative protection, for preventing and treating ROS-related diseases and conditions. The present invention addresses this and other related needs.

### BRIEF SUMMARY OF THE INVENTION

In one aspect, the present invention provides a method for preventing oxidative injury to a cell. The method comprises the step of contacting the cell with a composition, which comprises a lipid and a recombinant nucleic acid that comprises a polynucleotide encoding an extracellular superoxide dismutase (EC-SOD) such that the EC-SOD is expressed by the cell.

In some embodiments, the nucleic acid is not in a viral vector. In a preferred embodiment, the lipid is a polycationic lipid. In another preferred embodiment, the lipid is polycationic acrylamide lipid (polyAL). In a further embodiment, the lipid is conjugated to asialofetuin (AF) or galactose. In some other embodiments, the composition further comprises cholesterol.

In some embodiments, the cell is a part of an organ, which may remain in the body of an animal or have been isolated, e.g., from a donor's body prior to being transplanted into a recipient's body. In a preferred embodiment, the organ is a liver or a section of a liver. In another preferred embodiment, the contacting step is performed in vivo. In some embodiments, the composition is delivered to the cell by intravenous injection. In some other embodiments, the contacting step is performed in vitro and the method further comprises the step of transplanting the organ into a recipient animal. In a preferred embodiment, the animal is a human.

In some embodiments, the polynucleotide sequence encodes the amino acid sequence of SEQ ID NO:3. In a preferred embodiment, the polynucleotide sequence is SEQ ID NO:2. In some embodiments, the nucleic acid further comprises a promoter, which directs the expression of the polynucleotide sequence. In one preferred embodiment, the promoter is the cytomegalovirus (CMV) promoter. In another preferred embodiment, the promoter is a tissue-specific promoter. In a further preferred embodiment, the tissue-specific promoter is a liver-specific promoter.

In another aspect, the present invention provides a composition that comprises a lipid and a recombinant nucleic acid. The recombinant nucleic acid comprises a polynucleotide sequence encoding an extracellular superoxide dismutase (EC-SOD).

In some embodiments, the recombinant nucleic acid is not in a viral vector. In some embodiments, the lipid is a polycationic lipid. In one preferred embodiment, the lipid is a polycationic acrylamide lipid (polyAL). In another embodiment, the lipid is conjugated to asialofetuin (AF) or galactose. In some other embodiments, the composition further comprises cholesterol.

In some embodiments, the polynucleotide sequence encodes the amino acid sequence of SEQ ID NO:3. In one preferred embodiment, the polynucleotide sequence is SEQ ID NO:2. In other embodiments, the nucleic acid further comprises a promoter, which directs the expression of the polynucleotide sequence. In a preferred embodiment, the promoter is the cytomegalovirus (CMV) promoter. In another preferred embodiment, the promoter is a tissue-specific promoter. In a further preferred embodiment, the tissue-specific promoter is a liver-specific promoter.

In an additional aspect, the present invention provides an isolated organ that has been contacted with a composition, which comprises a lipid and a recombinant nucleic acid. The nucleic acid comprises a polynucleotide sequence encoding an extracellular superoxide dismutase (EC-SOD) such that at least some cells of the organ express the EC-SOD. The expression of EC-SOD may last a varying amount of time, depending on the nature of the tissue and exposure to the lipid-EC-SOD composition.

In some embodiments, the polynucleotide sequence encodes the amino acid sequence of SEQ ID NO:3. In a preferred embodiment, the polynucleotide sequence is SEQ ID NO:2. In some other embodiments, the nucleic acid further comprises a promoter, which directs the expression of the polynucleotide sequence. In one preferred embodiment, the promoter is the cytomegalovirus (CMV) promoter. In another preferred embodiment, the promoter is a tissue-specific promoter. In a further preferred embodiment, the tissue-specific promoter is a liver-specific promoter. In yet other embodiments, the organ is a liver or a section of a liver.

### BRIEF DESCRIPTION OF THE DRAWINGS

FIG. 1 SOD activity in EC-SOD plasmid-transfected Hep G₂ and Hep 3B cells. A. Hep G₂ cells were transfected with either the control plasmid (pEFGP-C1) or EC-SOD plasmid (PEGFP-C1-ECSOD) by Fugene 6. One day after the transfection, culture medium was changed to serum-free medium, and SOD activity in medium was measured 72 hours after the transfection. B. Hep 3B cells were transfected with either pEGFP-C1 or pEGFP-C1-ECSOD by our polycationic liposomes (PCL-Chol) as described in the Materials and Methods section. Two to three days after the transfection, the cells were subjected to geneticin (G418) selection. Geneticin-resistant cells were expanded and SOD activity in culture medium and cell lysates was determined spectrophotometrically one to three days after culture medium was changed to serum-free medium. The data are summarized from three experiments (**p<0.01 compared to control plasmid-transfected Hep 3B cells).

FIG. 2 Protection against superoxide-induced cell death by EC-SOD plasmid transfection. A. Hep G₂ cells were transfected with either the control plasmid (pEGFPC1) or EC-SOD plasmid (pEGFP-C1-ECSOD) by Fugene 6. One day after the transfection, the cells were first treated with buthionine sulfoximine (BSO) for 18 hours , then subsequently exposed to hypoxanthine (HX, 1 mM) and xanthine oxidase (XO, 2 milli-units) for 4-7 hours. Lactate dehydrogenase (LDH) leakage was measured to assess necrosis. The data are a mean of triplicates of one representative experiment (**p<0.01 compared to untransfected controls or cells transfected with pEGFP-C1). B. Hep G₂ cells were transfested with either control plasmid or EC-SOD plasmid, and were exposed to BSO plus HX/XO. An in situ apoptosis detection kit was employed to stain apoptotic (Apoptag-positive) cells in chamber slides after fixation. Apoptag-positive cells in 10 random fields were counted, and expressed as a percentage of total cells. The data are summarized from three independent experiments. **p<0.01 compared to untransfected controls or pEGFP-C1-transfected cells.

FIG. 3 Representative images of Apoptag-positive cells after exposure to hydroxyethyl radicals. An in situ apoptosis detection kit was employed to stain apoptotic (Apoptagpositive) cells in chamber slides after fixation. The experimental protocol is the same as FIG. 2B. Images A, C, and E are Apoptag-positive cells shown in red by staining with an in situ apoptosis detection kit; B, D, and F are the plain images of the same field. A and B, untransfected; C and D, control plasmid-transfected; E and F, EC-SOD plasmid-transfected. G. Apoptag-positive cells in 10 random fields were counted, and expressed as a percentage of total cells. **p<0.01 compared to untransfected controls or pEGFP-C1-transfected cells.

FIG. 4 Serum ALT levels in mice exposed to D-galactosamine (GalN)/lipopolysaccharide (LPS) with or without PCL-Chol-mediated EC-SOD gene delivery. The experimental protocol was described in detail in later sections. Data are summarized from three independent experiments. **p<0.01 compared to saline control, liposomes (PCL-Chol) alone, or transfected with pEGFP-C1.

FIG. 5 Representative micrographs of liver histology. Liver sections were fixed in 10% buffered formalin, embedded in paraffin, and stained by hematoxylin and eosin. A. GalN/LPS-treated with saline control; B. GalN/LPS-treated with liposomes (PCL-Chol) alone; C. GalN/LPS-treated plus PCL-Chol-mediated control plasmid (pEGFP-C1) transfer; and D. GalN/LPS-treated plus PCL-Chol-mediated EC-SOD gene (pEGFP-C1-ECSOD) transfer (100×).

FIG. 6 Representative GFP images in the liver of mice receiving PCL-Chol-mediated GFP plasmid transfer. Frozen liver tissues were collected when animals were sacrificed (two days after liposome-mediated gene delivery via portal vein injection), sectioned with a Crystat, and fixed in 10% buffered formalin. GFP images were recorded in a fluorescent microscope with a digital camera (100×). A. GalN/LPS-treaed with saline control; B. GalN/LPS-treated with liposomes (PCL-Chol) alone; C. GalN/LPS-treated plus PCL-Chol-mediated control plasmid (PEGFP-C1) transfer; and D. GalN/LPS-treated plus PCL-Chol-mediated EC-SOD gene (pEGFP-C1-ECSOD) transfer.

FIG. 7 Human EC-SOD gene expression in mouse liver and serum SOD activity in mice receiving liposome-mediated EC-SOD gene delivery. A. Human EC-SOD mRNA levels in mouse liver tissue were determined by real time quantitative RT-PCR using mouse β-actin as a house-keeping gene control. The relative human EC-SOD gene expression levels in three other groups were calculated based on an average level in the control group. B. Serum SOD activity after PCL-Chol-mediated EC-SOD gene delivery and subsequent GalN/LPS toxicity. Serum total SOD activity was measured spectrophotometrically two days after portal vein injection of liposomes or lipoplexes and subsequent GalN/LPS toxicity. *p<0.05, **p<0.01 compared to the three other groups.

FIG. 8 Liver glutathione content and lipid peroxidation after EC-SOD gene overexpression and GalN/LPS toxicity. A. Glutathione (GSH) content in the liver after portal vein injection of liposomes or lipoplexes and subsequent GalN/LPS toxic challenge. Liver GSH content was determined spectrophotometrically and expressed as nmol/mg protein of the tissue. The data are summarized from three experiments. *p<0.05 compared to other groups. B. Malondialdehyde (MDA) and 4-hydroxyalkenal (HAE), in the mouse liver after liposome-mediated EC-SOD gene delivery and subsequent GalN/LPS toxic challenge. Liver MDA and HAE contents were measured spectrophotometrically and expressed as nmol/mg protein of the tissue. Data are summarized from three independent experiments. *p<0.05 compared to the two other groups.

### DEFINITIONS

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) as well as intervening sequences (introns) between individual coding segments (exons).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 91%, 92%, 93%, 94%, 95%, 96%, 97%, 98%, 99%, or higher identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman, Adv. Appl. Math. 2:482 (1970), by the homology alignment algorithm of Needleman and Wunsch, J. Mol. Biol. 48:443-453 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. Nuc. Acids Res. 25:3389-3402 (1977), and Altschul et al. J. Mol. Biol. 215:403-410 (1990), respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “lipid” as used herein encompasses all lipids as conventionally defined, their derivatives (such as phospholipids and glycolipids), as well as any microscopic structure formed by lipid layers (such as the spherical structure of a liposome). This term further encompasses homogeneous or heterogeneous polymers of lipids. In the case of a heterogeneous polymer, different lipid monomers may vary in their physical characteristics (e.g., molecular structure and charge profile).

The term “viral vector” refers to a recombinant viral particle or a recombinant nucleic acid that comprises at least one viral gene encoding for a viral polypeptide, which may be genetically modified. A recombinant nucleic acid is not a “viral vector” merely because of the presence of a viral promoter and/or enhancer.

“Extracellular superoxide dismutase” or “EC-SOD” as used herein refers to an extracellular form of the three genetically distinct mammalian isoforms of superoxide dismutase (SOD). SOD is an enzymatic antioxidant and provides a defense mechanism against oxidative damage by mediating the disproportionation or dismutation of superoxide free-radical anion species:

.O⁻₂+.O⁻₂+2H⁺→H₂O₂+O₂

In contrast to SOD₁, a copper- and zinc-containing SOD (CuZn-SOD) localized primarily to cytoplasmic and nuclear compartments, and SOD₂, a manganese-containing SOD (Mn-SOD) found predominantly in mitochondria, SOD₃ (EC-SOD) is the predominant extracellular antioxidant enzyme. EC-SOD has been found in serum and in cerebrospinal, ascitic, and synovial fluids. In some human tissues such as uterus, umbilical cord, placenta, and arteries, EC-SOD enzyme activity equals or exceeds that of CuZn-SOD and Mn-SOD. An EC-SOD-like activity has been identified in every mammal examined thus far. Human EC-SOD (EC 1.15.1.1) is a secretory, homotetrameric glycoprotein with a molecular weight of about 135 kDa. As used herein, “EC-SOD” refers to any mammalian EC-SOD, including its naturally occurring or genetically modified variants that have detectable enzymatic activity in an in vitro SOD assay.

The term “organ” refers to a differentiated structure (as a heart, lung, kidney, or liver) consisting of cells and tissues and performing some specific function in an organism. This term also encompasses bodily parts performing a function or cooperating in an activity (e.g., an eye and related structures that make up the visual organs). The term “organ” further encompasses any partial structure of differentiated cells and tissue that is potentially capable of developing into a complete structure (e.g., a section of a liver).

### DETAILED DESCRIPTION OF THE INVENTION

### I. Introduction

The present invention provides, for the first time, an effective, lipid-based transfection method for introducing an enzymatic antioxidant, EC-SOD, to target cells for the purpose of treating or preventing oxidative injury to the cells.

### II. General Recombinant Technology

This invention relies on routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Ausubel et al. eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Polynucleotides encoding known genes are often available through commercial suppliers. Polyonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

### III. Construction of a Recombinant Nucleic Acid Comprising a Polynucleotide Sequence Encoding EC-SOD

### A. Obtaining an EC-SOD Coding Sequence

1. Cloning and Subcloning

A number of polynucleotide sequences encoding extracellular superoxide dismutases (EC-SOD), e.g., SEQ ID NO:2, have been previously determined and may thus be chemically synthesized or obtained from a commercial supplier.

The rapid progress in the studies of human genome has made possible a cloning approach where a human DNA sequence database can be searched for an allelic variant by screening for any gene segment that has a certain percentage of sequence homology to a known human EC-SOD coding sequence, e.g., SEQ ID NO:2. Any DNA sequence so identified can be subsequently obtained by chemical synthesis and/or polymerase chain reaction (PCR) such as overlap extension method. For a short sequence, completely de novo synthesis may be sufficient; whereas further isolation of full length coding sequence from a cDNA or genomic library using a synthetic probe may be necessary to obtain a larger gene.

Alternatively, a nucleic acid sequence encoding an EC-SOD can be isolated from a cDNA or genomic DNA library using standard cloning techniques such as polymerase chain reaction (PCR), where homology-based primers can often be derived from a known nucleic acid sequence encoding an EC-SOD. Most commonly used techniques for this purpose are described in, e.g., Sambrook and Russell, supra.

cDNA libraries suitable for obtaining coding sequence for an EC-SOD may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25:263-269 (1983); Ausubel et al., supra). Upon obtaining an amplified segment of nucleotide sequence by PCR, the segment can be further used as a probe to isolate the full length polynucleotide sequence encoding the EC-SOD from the cDNA library. General description of the procedure can be found in Sambrook and Russell, supra.

A similar procedure can be followed to obtain a full length sequence encoding an EC-SOD, e.g., SEQ ID NO:2, from a genomic library. Genomic libraries may be commercially available or can be constructed according to methods described in various scientific literature. In general, to construct a genomic library, the DNA is first extracted from an organism where an EC-SOD is likely found, and either mechanically sheared or enzymatically digested to yield fragments of about 12-20 kb in length. The fragments are then separated by gradient centrifugation from undesired sizes and are constructed in bacteriophage λ vectors. These vectors and phages are packaged in vitro. Recombinant phages are analyzed by plaque hybridization as described in Benton and Davis, Science, 196:180-182 (1977). Colony hybridization is carried out as described by Grunstein et al., Pro. Natl. Acad. Sci. USA, 72:3961-3965 (1975).

Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library. Using the segment as a probe, the full length nucleic acid encoding an EC-SOD can be obtained subsequently.

Using the same general cloning methodology described above, one skilled artisan can also obtain a nucleic acid encoding an EC-SOD from a different species, e.g., a non-human mammal, based on sequence homology to a previously identified human EC-SOD coding sequence, e.g., SEQ ID NO:2. Some examples of known mammalian EC-SOD coding sequences include those described in, e.g., GenBank Accession Nos. BC010975 (mouse), X94371 (rat), and Y13339 (rabbit).

2. EC-SOD Variants

Any EC-SOD variant that retains the same enzymatic activity of the wild-type protein is useful for practicing the present invention. For example, Sandstrom et al., J. Biol. Chem., 269:19163-19166 (1994) describes a human EC-SOD variant that contains an Arg213 to Gly213 substitution and yet retains full enzymatic activity (coding sequence described in GenBank Accession No. S715447). EP 0 593 435 discloses additional known human EC-SOD variants or mutants.

Furthermore, to achieve improved characteristics of a recombinantly produced EC-SOD, such as enhanced enzymatic activity and resistance to degradation, modifications can also be made to an EC-SOD polynucleotide coding sequence, whether such sequence is naturally-occurring or generated in vitro.

A variety of protocols have been established and described in the art for the purpose of introducing diversity into a polypeptide. See, e.g., Zhang et al., Proc. Natl. Acad. Sci. USA, 94:4504-4509 (1997); and Stemmer, Nature, 370:389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortle, Science, 229:1193-1201 (1985)), mutagenesis using uracil-containing templates (Kunkel, Proc. Natl. Acad. Sci. USA, 82:488-492 (1985)), oligonucleotide-directed mutagenesis (Zoller and Smith, Nucl. Acids Res., 10:6487-6500 (1982)), phosphorothioate-modified DNA mutagenesis (Taylor et al., Nucl. Acids Res., 13:8749-8764 and 8765-8787 (1985)), and mutagenesis using gapped duplex DNA (Kramer et al., Nucl. Acids Res., 12:9441-9456 (1984)).

Other suitable methods include point mismatch repair (Kramer et al., Cell, 38:879-887 (1984)), mutagenesis using repair-deficient host strains (Carter et al., Nucl. Acids Res., 13:4431-4443 (1985)), deletion mutagenesis (Eghtedarzadeh and Henikoff, Nucl. Acids Res., 14:5115 (1986)), restriction-selection and restriction-purification (Wells et al., Phil. Trans. R. Soc. Lond. A, 317:415-423 (1986)), mutagenesis by total gene synthesis (Nambiar et al., Science, 223:1299-1301 (1984)), double-strand break repair (Mandecki, Proc. Natl. Acad. Sci. USA, 83:7177-7181 (1986)), mutagenesis by polynucleotide chain termination methods (U.S. Pat. No. 5,965,408), and error-prone PCR (Leung et al., Biotechniques, 1:11-15 (1989)).

Diversity also can be generated in nucleic acids or populations of nucleic acids using a recombinational procedure termed “incremental truncation for the creation of hybrid enzymes” (“ITCHY”) described in Ostermeier et al., Nature Biotech., 17:1205 (1999). This approach can be used to generate an initial library of variants which can optionally serve as a substrate for one or more in vitro or in vivo recombination methods. See, also, Ostermeier et al., Proc. Natl. Acad. Sci. USA, 96:3562-3567 (1999); Ostermeier et al., Bio. Me. Chem., 7:2139-2144 (1999).

By using the methods described above, a number of nucleic acids encoding EC-SOD variants can be derived from the wild-type sequences or in vitro generated sequences encoding EC-SOD. Since not all diversity is functional, the recombinant EC-SOD variants should be screened for their enzymatic activity in assays described in a later section.

Upon acquiring a nucleic acid sequence encoding an EC-SOD, e.g., SEQ ID NO:2, modifications to the coding sequence, e.g., substitutions, insertions, or deletions, may be subsequently made to optimize the enzyme's activity. Alternatively, the polynucleotide sequence encoding an EC-SOD can be altered to coincide with the preferred codon usage of a particular host cell. Upon completion of the modification, the coding sequence can be subcloned into a suitable vector, for instance, an expression vector (e.g., pEGFP-C1 or pIRES2-EGFP), so that a recombinant EC-SOD can be produced from the construct.

### B. Recombinant Nucleic Acid Comprising EC-SOD Coding Sequence

Expression Vectors

The nucleic acid encoding an EC-SOD is typically cloned into an intermediate vector before transformation into prokaryotic or eukaryotic cells for replication and/or expression. The intermediate vector is typically a prokaryote vector such as a plasmid or shuttle vector.

To obtain high level expression of a cloned gene, such as the cDNA encoding an EC-SOD (e.g., SEQ ID NO:2), one typically subclones the cDNA into an expression vector that contains a strong promoter to direct transcription, a transcription/translation terminator, and a ribosome binding site for translational initiation. Numerous expression vectors utilizing prokaryotic or eukaryotic promoters are well known in the art and fully described in scientific literature such as Sambrook and Russell, supra, and Ausubel et al, supra. Viral promoters (e.g., cytomegalovirus, or CMV, promoter) are useful for the expression of EC-SOD. A tissue-specific promoter directing gene expression in a particular target cell type (e.g., hepatocytes) is particularly useful for the present invention. For example, hepatocyte-specific promoter alpha-1-antitrypsin promoter can be used for EC-SOD delivery and expression in the liver. EC-SOD expression may be driven by either a constituent or inducible promoter.

Selection of the promoter used to direct expression of a heterologous nucleic acid (e.g., SEQ ID NO:2) depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for expressing an EC-SOD in host cells. A typical expression cassette thus contains a promoter operably linked to the nucleic acid sequence encoding an EC-SOD and signals required for efficient polyadenylation of the transcript, ribosome binding sites, and translation termination. Additional elements of the cassette may include enhancers and, if genomic DNA is used as the structural gene, introns with functional splice donor and acceptor sites.

Furthermore, the expression cassette should also contain a transcription termination region downstream of the EC-SOD gene to provide for efficient termination. The termination region may be obtained from the same gene as the promoter sequence or may be obtained from different genes.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase and dihydrofolate reductase. Alternatively, high-yield expression systems not involving gene amplification are also suitable.

The elements that are typically included in expression vectors may also include a replicon that functions in E. coli, a gene encoding antibiotic resistance to permit selection of cells that harbor recombinant plasmids, and unique restriction sites in nonessential regions of the plasmid to allow insertion of exogenous sequences. The particular antibiotic resistance gene chosen is not critical, any of the many resistance genes known in the art are suitable. The prokaryotic sequences are preferably chosen such that they do not interfere with the replication of the DNA in eukaryotic cells.

### III. Target Cells for Recombinant Expression of EC-SOD

Cells of various tissue types, both epithelial and endothelial, are suitable targets for the expression of a recombinant EC-SOD of the present invention (e.g., a polypeptide comprising an amino acid sequence of SEQ ID NO:3). See descriptions in numerous scientific publications such as Sambrook and Russell, supra. According to the present disclosure, a recombinant EC-SOD is preferably expressed in a eukaryotic cell. Cells of many different tissue types, such as liver, kidney, or other organs, are suitable for receiving and expressing a polynucleotide sequence encoding an EC-SOD.

### IV. Transfection Methods

Standard transfection methods can be used to produce a recombinant EC-SOD in, e.g., eukaryotic cells of a certain tissue type (such as epithelial or endothelial cells) or as a part of an organ (such as liver or kidney) (see, e.g., Colley et al., J. Biol. Chem., 264:17619-17622 (1989); Guide to Protein Purification, in Methods in Enzymology, vol. 182 (Deutscher, ed.), 1990). In general, transfection vectors are classified into two categories, viral or non-viral.

There are many well-known procedures for introducing foreign nucleotide sequences into target cells. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA, or other foreign genetic material into a host cell (see, e.g., Sambrook and Russell, supra). Due to the side-effects associated with gene delivery mediated by viral vectors, such as host genome integration, insertion-induced mutation, and immune responses to viral components, the non-viral delivery methods are preferred for practicing the present invention.

A preferred method for practicing the present invention is a liposome-based method for introducing an EC-SOD gene into target cells for expression. Art-recognized liposomes include emulsions, foams, micelles, insoluble monolayers, liquid crystals, phosphlipid dispersion, lamellart layers and the like. Liposomes for use in the present invention are formed from standard vesicle-forming lipids, which generally include neutral or negatively charged phospholipids and a sterol (such as cholesterol). The selection of lipids is generally guided by consideration of, e.g., liposome size, acid lability, and stability of the liposomes in the environment of the host cells (e.g., in the culture medium or blood stream). A variety of methods are known in the art for preparing liposomes, as described in, e.g., Szoka et al., Ann. Rev. Biophys. Bioeng., 9:467 (1980); U.S. Pat. Nos. 4,235,871; 4,501,728; 4,837,028, and 5,019,369. In one preferred embodiment of the present invention, liposomes are formed from polycationic lipids using the technique described in Wu et al., Bioconj. Chem., 12:251-257 (2001) and Liu et al., Gene Ther., 10:180-187 (2003). This formulation of polycationic liposomes has the advantages of non-toxicity, high stability in the blood stream, and high efficacy for both in vitro and in vivo gene delivery.

Many formulations of liposomes as gene transfer agents are used for in vitro and in vivo gene transfection (Wu et al., Exp. Opin. Invest. Drugs, 7:1795-1817 (1998); Niidome et al., Gene Ther., 9:1647-1652 (2002)). With modifications in lipid structure, formulations, and targeting approaches, significant improvements in gene transfer efficacy have been achieved (Templeton et al., Nature Biotechnol., 15:647-652 (1997); Dasi et al., J. Mol. Med., 79:205-212 (2001); Liu et al., Gene Ther., 4:517-523 (1997); Wu et al., Front Biosci., 7:d717-725 (2002); Kren et al., Proc. Natl. Acad. Sci. USA, 96:10349-10354 (1999)). A series of successful attempts in genetic correction (Dasi et al., J. Mol. Med., 79:205-212 (2001); Kren et al., Proc. Natl. Acad. Sci. USA, 96:10349-10354 (1999)), cancer gene therapy (Mohr et al., Human Gene Ther., 12:799-809 (2001)) and targeting liver gene delivery (Kawakami et al., Pharm. Res., 17:306-313 (2000)), have been reported.

### V. Preparation of EC-SOD-PCL Complex

### A. Preparation of PCL

In some embodiments of the present invention, a polycationic lipid (PCL) is used to formulate polycationic liposomes with cholesterol (Chol) (Wu et al., Bioconjugate Chem., 12:251-257 (2001)). The liposomal formulations such as the PCL-Chol formulation are non-toxic, bind the least to plasma proteins, and displays high gene transfer efficacy to the liver when compared to the commonly used (1,2-bis(dioleoyloxy)-3-(trimethylamonio)propane-cholesterol (DOTAP-Chol) or DOTAP-L-α dioleoyl phosphatidylethanolamine (DOPE) formulations (Liu et al., Gene Ther., 10:180-187 (2003)).

The PCL used for practicing the present invention can be prepared from cationic lipid monomers. Chemical methods for cationic lipid polymerization are known in the art. For example, Wu et al., Bioconj. Chem., 12:251-257 (2001) describes polymerization of a cationic acrylamide lipid (AL) to form a PCL that can be used in liposome-mediated gene transfer. Further description of using this PCL for in vivo gene delivery to mouse liver can be found in Liu et al., Gene Ther., 10:180-187 (2003).

Various other cationic lipid monomers may be used in a similar polymerization process to produce PCL. In essence, the polar domain of a cationic transfection agent can be modified to incorporated a functional moiety for polymerization (e.g., acrylamide) and prepare PCL by polymerization (e.g., by polymerization of headgroup acrylamide or acrylate functionality). Any acrylamide derivative [—NRC(O)CR′═CH₂] of a known cationic lipid may be used for practicing the present invention.

In some cases, only one type of cationic lipid monomers (e.g., AL) is involved in producing a homopolymeric PCL. In other cases, a mixture of two or more different cationic lipid monomers may be used to produce a PCL, which is a heteropolymer comprising different monomers at varying ratios.

### B. PCL-Nucleic Acid Complex

PCL-nucleic acid formulations are also described in Liu et al., Gene Ther., 10:180-187 (2003). In some embodiments, a PCL-nucleic acid formulation further includes cholesterol (Chol). In one particular embodiment, PCL and cholesterol are mixed at a 3:1 ratio to produce PCL-Chol liposomes, which are further complexed with an expression construct encoding human EC-SOD at a charge ratio of 5:1. Varying ratios of PCL, nucleic acid, and if applicable, cholesterol, may be used for generating the lipid complex, depending on the nature of the PCL and the target cell or tissue type. One of skill in the art will be able to determine the optimal ratio(s) through routine experimentation.

### C. Conjugation of Sugar to PCL for Targeting Specific Tissue of Interest

In order to facilitate the targeting of specific tissue for EC-SOD delivery, certain oligosaccharide chains may be conjugated to a lipid monomer or PCL. For example, an asialofetuin (AF) or galactose residue may be used as a lipid conjugate for liver-specific delivery. The chemical methods for such conjugation are known in the art and also described in the scientific literature, e.g., Wu et al., Hepatology, 27:772-778 (1998) describes a method for conjugating AF to PCL; Yoon et al., Biotechnology and Bioengineering, 78:1 (2002) describes a method for conjugating sugar (such as galactose) residues to PCL.

### VI. Delivering EC-SOD Nucleic Acid-PCL Complex to Target Cells

Upon completion of preparing a lipocomplex that includes an expression vector containing an EC-SOD coding sequence and a polycationic lipid, the complex is administered to target cells or a target organ for protection against oxidative injuries.

### A. In Vivo Delivery

The lipid-EC-SOD compositions of the present invention are suitable for use in a variety of drug delivery systems. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990).

The compositions are intended for parenteral, intranasal, topical, oral, or local administration, such as by aerosol or transdermally, for prophylactic and/or therapeutic treatment. Commonly, the compositions are administered parenterally, e.g., intravenously. Thus, the invention provides compositions for parenteral administration, which comprise the EC-SOD gene and PCL dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents, and the like.

Methods for in vivo delivery of an EC-SOD gene according to the present invention include various applications of systemic or topical in nature, depending the cell or tissue type and anatomic location. For example, intravenous injection may be suitable for delivering EC-SOD to cells or tissue easily accessible, e.g., located at a highly vascularized area such as lungs. Direct injection to an organ is desirable in some cases. For example, a compound circulating in a patient's blood stream cannot easily access the brain tissue due to the blood-brain barrier. Thus, an effective delivery to the brain may be accomplished by direction injection of a lipocomplex comprising an EC-SOD gene into the brain chamber, or by injection into the spinal cord for delivery via cerebralspinal fluid circulation.

The compositions of the present invention may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 and 8.

The compositions containing a lipid and a recombinant EC-SOD coding sequence can be administered for prophylactic and/or therapeutic treatments. In therapeutic applications, compositions are administered to a patient already suffering from a disease or condition related to oxidative injury, in an amount sufficient to cure or at least partially arrest the symptoms of the disease and its complications. An amount adequate to accomplish this is defined as a “therapeutically effective dose.” Amounts effective for this use will depend on the severity of the disease or condition and the weight and general state of the patient, but generally range from about 0.5 mg to about 2,000 mg of the expression construct comprising an EC-SOD gene per day or every other day for a 70 kg patient, with dosages of from about 5 mg to about 500 mg of the construct per day or every other day being more commonly used.

In prophylactic applications, compositions containing the lipid-EC-SOD gene complex of the invention are administered to a patient susceptible to or otherwise at risk of a particular disease/condition related to oxidative injury. Some examples of such situations include ingestion of certain medication (such as acetaminophen) or alcohol or exposure of the lungs to a high concentration of oxygen. Such an amount is defined to be a “prophylactically effective dose.” In this use, the precise amounts again depend on the patient's state of health and weight, but generally range from about 0.5 mg to about 1,000 mg per 70 kilogram patient, more commonly from about 5 mg to about 500 mg per 70 kg of body weight. The frequency of administration ranges from once per day to once every two to three days.

The frequency and duration for delivering the compositions comprising EC-SOD-PCL complex depend on the nature of the oxidative injury or the risk of such an injury. If the injury or risk sough to address is acute in nature, e.g., acute alcohol toxicity to liver, a single administration may be sufficient; on the other hand, if the injury or risk is chronic in nature, e.g., prolonged exposure to ROS, multiple administrations may be necessary. A possible treatment schedule is one administration every two to three days, for a time period as determined proper by a treating physician.

In any event, the compositions should provide a quantity of the recombinant nucleic acid encoding EC-SOD to allow sufficient expression of the enzyme and to effectively treat the patient.

### B. In Vitro or Ex Vivo Delivery

The compositions comprising a lipid and a recombinant EC-SOD expression construction as described in the present application are also useful in an in vitro or ex vivo procedure, e.g., for preparing or preserving an isolated donor organ prior to transplantation.

To prevent oxidative damage to an isolated organ prior to its re-implantation or transplantation into a patient's body, the compositions of the present invention, which comprises EC-SOD-lipid complex, can be included as a part of a perfusion solution used to, for example, flush blood out of a donor organ and preserve the organ for transport and a later transplantation. For example, perfusing a donor liver and retaining the perfusion solution in the organ are the routine procedure before the transplantation into a recipient. A number of perfusion solutions are known in the art, including the liver perfusion solution of the University of Wisconsin and its modified versions (available from ViaSpan Laboratories, Pamona, N.Y.), as well as Custodiol HTK solution by Odyssey Pharmaceuticals, Inc. (East Hanover, N.J.).

After an EC-SOD gene is delivered to the target cells, the cells are placed under conditions favoring expression of the recombinant EC-SOD, which may be confirmed using immunological or enzymatic assays identified below.

### VII. Detection of EC-SOD Expression

Following the transfection procedure, the expression of recombinant EC-SOD may be confirmed by various assays well known among those skilled in the art.

First, gene expression can be detected at nucleic acid level. A variety of methods of specific DNA and RNA measurement using nucleic acid hybridization techniques are commonly used (e.g., Sambrook and Russell, supra). Some methods involve an electrophoretic separation (e.g., Southern blot for detecting DNA and Northern blot for detecting RNA), but detection of DNA or RNA can be carried out without electrophoresis as well (such as by dot blot, or in situ hybridization if the detection is made within a target tissue). The presence of nucleic acid encoding a recombinant EC-SOD in transfected cells can also be detected by PCR or PCR-based methods (e.g., real-time PCR and RT-PCR) using sequence-specific primers.

Second, recombinant EC-SOD expression can be detected at the polypeptide level. As EC-SOD is a secreted protein, both extracellular material (e.g., extracellular matrix) and cytoplasmic material (e.g., cell lysate) may be examined for the presence of EC-SOD protein. Various immunological assays (such as enzyme-linked immune absorbent assay (ELISA), Western blot, and immunohistochemistry) are routinely used by those skilled in the art to measure the level of a gene product, particularly using polyclonal or monoclonal antibodies that react specifically with a recombinant EC-SOD of the present invention, such as a polypeptide comprising the amino acid sequence of SEQ ID NO:3, (e.g., Harlow and Lane, Antibodies, A Laboratory Manual, Chapter 14, Cold Spring Harbor, 1988; Kohler and Milstein, Nature, 256:495-497 (1975)). Such techniques require antibody preparation by selecting antibodies with high specificity against the recombinant EC-SOD or an antigenic portion thereof. The methods of raising polyclonal and monoclonal antibodies are well established and their descriptions can be found in the literature, see, e.g., Harlow and Lane, supra; Kohler and Milstein, Eur. J. Immunol., 6:511-519 (1976).

In addition, functional assays may also be performed for detecting the recombinant EC-SOD of the presenting invention, e.g., a polypeptide having an amino acid sequence of SEQ ID NO:3, in transfected cells or tissue. Assays for detecting the enzymatic activity of an EC-SOD are generally described in a later section.

### VIII. Immunoassays for Detection of EC-SOD Expression

To confirm the production of a recombinant EC-SOD, immunological assays may be useful to detect in a sample the expression of this enzyme. Immunological assays are also useful for quantifying the expression level of the recombinant EC-SOD.

### A. Production of Antibodies Against EC-SOD

Methods for producing polyclonal and monoclonal antibodies that react specifically with an immunogen of interest are known to those of skill in the art (see, e.g., Coligan, Current Protocols in Immunology Wiley/Greene, NY, 1991; Harlow and Lane, Antibodies: A Laboratory Manual Cold Spring Harbor Press, NY, 1989; Stites et al. (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y., 1986; and Kohler and Milstein Nature 256: 495-497, 1975). Such techniques include antibody preparation by selection of antibodies from libraries of recombinant antibodies in phage or similar vectors (see, Huse et al., Science 246: 1275-1281, 1989; and Ward et al., Nature 341: 544-546, 1989).

In order to produce antisera containing antibodies with desired specificity, the polypeptide of interest (e.g., a human EC-SOD) or an antigenic fragment thereof can be used to immunize suitable animals, e.g., mice, rabbits, goat, horse, or monkey. A standard adjuvant, such as Freund's adjuvant, can be used in accordance with a standard immunization protocol. Alternatively, a synthetic antigenic peptide derived from that particular polypeptide can be conjugated to a carrier protein and subsequently used as an immunogen.

The animal's immune response to the immunogen preparation is monitored by taking test bleeds and determining the titer of reactivity to the antigen of interest. When appropriately high titers of antibody to the antigen are obtained, blood is collected from the animal and antisera are prepared. Further fractionation of the antisera to enrich antibodies specifically reactive to the antigen and purification of the antibodies can be performed subsequently, see, Harlow and Lane, supra, and the general descriptions of protein purification provided above.

Monoclonal antibodies are obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519, 1976). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and the yield of the monoclonal antibodies produced by such cells may be enhanced by various techniques, including injection into the peritoneal cavity of a vertebrate host.

Additionally, monoclonal antibodies may also be recombinantly produced upon identification of nucleic acid sequences encoding an antibody with desired specificity (e.g., specifically recognizing a human EC-SOD) or a binding fragment of such antibody by screening a human B cell cDNA library according to the general protocol outlined by Huse et al., supra. The general principles and methods of recombinant polypeptide production discussed above are applicable for antibody production by recombinant methods.

### B. Immunoassays for Detecting EC-SOD Expression

Once antibodies specific for an EC-SOD are available, the amount of the enzyme in a sample, e.g., a cell lysate, a small section of tissue, a sample of blood or other body fluids such as saliva, cerebrospinal fluid, join fluid, etc., can be measured by a variety of immunoassay methods (such as ELISA or Western blot) providing qualitative and quantitative results to a skilled artisan. For a review of immunological and immunoassay procedures in general see, e.g., Stites, supra; U.S. Pat. Nos. 4,366,241; 4,376,110; 4,517,288; and 4,837,168.

1. Labeling in Immunoassays

Immunoassays often utilize a labeling agent to specifically bind to and label the binding complex formed by the antibody and the target protein (e.g., a human EC-SOD). The labeling agent may itself be one of the moieties comprising the antibody/target protein complex, or may be a third moiety, such as another antibody, that specifically binds to the antibody/target protein complex. A label may be detectable by spectroscopic, photochemical, biochemical, immunochemical, electrical, optical or chemical means. Examples include, but are not limited to, magnetic beads (e.g., Dynabeadsm), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase, and others commonly used in an ELISA), and calorimetric labels such as colloidal gold or colored glass or plastic (e.g., polystyrene, polypropylene, latex, etc.) beads.

In some cases, the labeling agent is a second antibody bearing a detectable label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111:1401-1406 (1973); and Akerstrom, et al., J. Immunol., 135:2589-2542 (1985)).

2. Immunoassay Formats

Immunoassays for detecting a target protein of interest (e.g., a recombinant human EC-SOD) from samples may be either competitive or noncompetitive. Noncompetitive immunoassays are assays in which the amount of captured target protein is directly measured. In one preferred “sandwich” assay, for example, the antibody specific for the target protein can be bound directly to a solid substrate where the antibody is immobilized. It then captures the target protein in test samples. The antibody/target protein complex thus immobilized is then bound by a labeling agent, such as a second or third antibody bearing a label, as described above.

In competitive assays, the amount of target protein in a sample is measured indirectly by measuring the amount of an added (exogenous) target protein displaced (or competed away) from an antibody specific for the target protein by the target protein present in the sample. In a typical example of such an assay, the antibody is immobilized and the exogenous target protein is labeled. Since the amount of the exogenous target protein bound to the antibody is inversely proportional to the concentration of the target protein present in the sample, the target protein level in the sample can thus be determined based on the amount of exogenous target protein bound to the antibody and thus immobilized. See, e.g., Karlson et al., Lab. Invest., 70:705-710 (1994).

In some cases, western blot (immunoblot) analysis is used to detect and quantify the presence of an EC-SOD in the samples. The technique generally comprises separating sample proteins by gel electrophoresis on the basis of molecular weight, transferring the separated proteins to a suitable solid support (such as a nitrocellulose filter, a nylon filter, or a derivatized nylon filter) and incubating the samples with the antibodies that specifically bind the target protein. These antibodies may be directly labeled or alternatively may be subsequently detected using labeled antibodies (e.g., labeled sheep anti-mouse antibodies) that specifically bind to the antibodies against the EC-SOD. See, e.g., Pineda et al., J. Neurotrauma, 18:625-634 (2001); Bowler et al., J. Biol. Chem., 277:16505-16511 (2002).

Various in situ immunochemical staining methods using antibodies against EC-SOD are also useful for demonstrating the presence of an EC-SOD in a tissue sample, e.g., from a target organ following EC-SOD delivery.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev., 5:34-41 (1986)).

### IX. Enzymatic Assays for EC-SOD Activity

To confirm the presence of a biologically active EC-SOD, in vitro enzymatic assays can be carried out. Various methods are known in the art for detecting SOD or EC-SOD activity, which may serve as a means of determining the efficacy of gene delivery.

Generally, total SOD enzymatic activity is measured by direct spectrophotometric methods employing an appropriate substrate, e.g., KO₂. See, Marklund, J. Biol. Chem., 251:7504-7507, (1976). Reagents known to selectively inactivate one or two isoforms of SOD may be used in a SOD assay to distinguish the enzymatic activity attributed to the individual isoforms of SOD. For example, cyanide is useful for distinguishing the cyanide-sensitive SOD, from EC-SOD and cyanide-resistant SOD₂. Furthermore, antibodies specifically recognize only one isoform of SOD (e.g., EC-SOD) but not the other two can be used to determine the specific enzymatic activity of this isotype by direct or indirect means. For more description of enzymatic assays for measuring SOD activity and EC-SOD specific activity, see, e.g., Marklund, J. Clin. Invest., 74:1398-1403 (1984); Marklund, in Handbook of Methods for Oxygen Radical Research (Greenwald, R., ed) pp. 249-255, CRC Press, Inc., Boca Raton, Fla. (1985); Sandstrom et al., supra; and Marklund, Methods Enzymol., 349:74-80 (2002).

### EXAMPLES

The following examples are provided by way of illustration only and not by way of limitation. Those of skill in the art will readily recognize a variety of non-critical parameters that could be changed or modified to yield essentially similar results.

### Materials and Methods

1. Subcloning EC-SOD Expression Plasmid

Plasmid pUC18-ECSOD, kindly provided by Dr. Stefan Marklund, Department of Medical Biosciences, Ume [symbol] University, Ume [symbol] , Sweden, (Hjalmarsson et al., Proc. Natl. Acad. Sci. USA, 84:6340-6344 (1987)) was digested by EcoRI, and the EC-SOD gene was ligated into pEGFP-C1 or pIRES2-EGFP from Clontech Laboratories, Inc. (Palo Alto, Calif.) at the multiple cloning site to form new plasmids, pEGFP-C1-ECSOD and pIRES2-EGFP-ECSOD. The orientation of the ligated fragment of the EC-SOD gene in the plasmids was verified by restriction enzymes (BamH I) and sequencing, using a specific forward primer. The resulting plasmid DNA was transformed into competent E. coli cells from Gibco Life Science Technologies (Grand Island, N.Y.). The plasmid DNA was extracted from overnight cultures of the competent cells and purified by affinity chromatography with an Endofree plasmid extraction kit from Qiagen, Inc. (Valencia, Calif.). The quality of the DNA was determined by UV spectroscopy and agarose gel electrophoresis (1.0% agarose gel with 0.5 mg/mL ethidium bromide) after cleavage by specific restriction endonucleases. The DNA concentration was quantitated spectrophotometrically. The plasmid DNA was frozen at −20° C., and diluted to 1 μg/μl in water for in vitro transfection, or in normal saline for in vivo gene delivery, prior to use (Wu et al., Bioconjugate Chem., 12:251-257 (2001)).

2. Transfection of EC-SOD Plasmids in Hep G₂ Cells and Measurement of SOD Activity in Culture Medium.

Hep G₂ cells were cultured in minimum essential medium (MEM) plus 10% bovine fetal serum (FBS) and antibiotics, and were transiently transfected with either the control plasmid (PEGPF-C1) or EC-SOD plasmid (PEGFP-C1-ECSOD) using Fugene 6 (Roche Molecular Biochemicals, Indianapolis, Ind.), when they were 50-70% confluent. One day after the transfection, the culture medium was changed to serum free medium. Culture medium was collected 48 hours after the medium change for the spectrophotometric determination of superoxide dismutase activity with a commercially available kit from Calbiochem Inc. San Diego, Calif. For stable transfection, Hep 3B cells were cultured with MEM plus 10% FBS and antibodies, and were transfected with either pEGFP-C1-ECSOD or control plasmids by a PCL-Chol formulation as we reported previously (Wu et al., Bioconjugate Chem., 12:251-257 (2001)). The transfected cells were subjected to geneticin (G418) selection (350 μg/ml medium) over one week. G418-resistant cells were grown in the medium containing G418 until it was changed for serum-free medium. One to three days after the cells were cultured in the serum-free medium, SOD activity in cell culture medium and cell lysates was determined by the kit mentioned above.

3. Necrosis and/or Apoptosis in Hep G₂ Cells Induced by Superoxide Anions or Hydroxyethyl Radicals (HER).

One day after Hep G₂ cells were transiently transfected with either the control plasmid or EC-SOD plasmid, the cells were subjected to pretreatment with a glutathione-depleting agent, buthionine sulfoximine (BSO, 0.3 mM) for 18 hours , and a subsequent exposure to a superoxide anion-generating system, hypoxanthine (HX, 1 mM) and xanthine oxidase (XO, 2 milli-units) for 4-7 hours (Gabriel et al., J. Hepatol., 29:614-627 (1998)). In separate experiments, the transfected cells were subsequently exposed to an HER-generating system, which consists of hydrogen peroxide (0.1 mM), ferrous ammonium sulfate (20 μM), and ethanol (200 mM) for 8 hours (Sakurai et al., Free Rad. Biol. Med., 28:2:273-280 (2000)).

After the cells were exposed to either superoxide anions or HER, culture medium was collected for the determination of lactate dehydrogenase (LDH) leakage (Roche Molecular Biochemicals, Indianapolis, Ind.), which is employed as an indicator of cell necrosis (Wu et al., Bioconjugate Chem., 12:251-257 (2001)). After the ROS exposure, the cells cultured on LabTech chamber slides (Fisher Scientific Inc. Santa Clara, Calif.) were fixed with 1% paraformaldehyde, and stained with an in situ apoptosis detecting kit, Apoptag, which uses rhodamine-conjugated anti-digoxigenin for final images (Intergen Company, Purchase, N.Y.).

4. Liposome Generation and Size Measurements

PCL was synthesized and validated as previously described (Wu et al., Bioconjugate Chem., 12:251-257 (2001)) and the PCL-Chol liposome formulation was generated in a molar ratio of 3:1 as described in detail previously (Liu et al., Gene Ther., 10:180-187 (2003)). The liposome size was measured after sonication by a laser-based, submicron particle size analyzer from Beckman Coulter, Inc., as described previously (Wu et al., Hepatology, 27:772-778 (1998); Wu et al., Bioconjugate Chem., 12:251-257 (2001)). The size of liposomes at different times of generation is between 200-250 nm before use. For animal experiments, PCL-Chol liposomes were complexed with plasmid DNA at a charge ratio of 5:1 (Liu et al., Gene Ther., 10:180-187 (2003)). For each mouse, 200 μl of liposome suspension containing 0.3 μmol PCL and 0.1 μmol cholesterol was injected via the portal vein.

5. GalN/LPS-Induced Acute Liver Injury and its Prevention by Polycationic Liposome-Mediated EC-SOD Gene Delivery.

Polycationic liposome-mediated EC-SOD gene delivery to the liver was conducted according to our previous description (Liu et al., Gene Ther., 10:180-187 (2003)). The animal experimental protocol was approved by the Animal Care and Use Administrative Advisory Committee of the University of California, Davis, according to guidelines of the National Institutes of Health. C57BL/6 mice, provided by Charles River Laboratory, Wilmington, Mass., were fed a pellet diet and water ad libitum, and kept on a 12 hour-light/dark cycle. Animals were randomly divided into four groups and all received one injection of thyroid hormone (triiodothyronine or T₃, 4 mg/kg, s.c.) in order to stimulate hepatocyte proliferation. Two days after T₃ injection, the animals were treated following a protocol outlined in Table 1. Animals were anesthetized with pentobarbital for the operation (60 mg/kg, i.p.). Polycationic liposomes alone (200 μl), or complexes with plasmid DNA (lipoplexes) were injected via the portal vein (100 μg of control plasmid pEGFP-C1 or pEGFP-C1-ECSOD per mouse). One day after the lipoplex injection, animals were exposed to GalN (500 m/kg, i.p.) plus LPS (25 μg/kg, i.p.). Both GalN and LPS were dissolved in normal saline. The animals were sacrificed 24 hours after the GalN/LPS intoxication, and the blood was collected for measurement of alanine aminotransferase (ALT) levels (Sigma Chemical Co. St. Louis, Mo.) and serum SOD activity as described previously. Part of the liver tissue was fixed in 10% buffered formalin for routine paraffin embedding and hematoxylin-eosine staining. Some liver tissue was snap-frozen for sectioning. The frozen sections were subsequently fixed in 10% buffered formalin and examined for green fluorescence protein (GFP) expression under a fluorescent microscope. The images were recorded with a digital videocamera (Wu et al., Bioconjugate Chem., 12:251-257 (2001)). The reduced form of glutatione (GSH), malondialdehyde (MDA) and 4-hydroxyalkenal (HAE) levels in the liver tissue were determined spectrophotometrically by commercially available kits (OXISResearch, Portland, Oreg.) according to the manufacture's manuals and expressed as nmol/mg protein in tissue.

TABLE 1
Design of animal experiment

| | | | GalN/LPS |
|---|---|---|---|
| | T₃ | Treatments | (500 mg + |
| | (4 mg/ | (Portal vein injection | 25 μg/ |
| Group | kg, s.c.) | of lipoplexes) | kg, i.p.) |
| Saline | + | Saline (300 μl) | + |
| Liposome | + | PCL-Chol (200 μl) | + |
| Liposome + Control | + | PCL-Chol + pEGFP-C1 | + |
| plasmid DNA | | (100 μg DNA) | |
| Liposome + EC-SOD | + | PCL-Chol + pEGFP-C1- | + |
| plasmid DNA | | ECSOD (100 μg DNA) | |

Time line of the experiment

| T₃ | | Lipoplexes | GalN/LPS | Sacrifice |
|---|---|---|---|---|
| ↓ | | ↓ | ↓ | ↓ |
| Day 0 | 1 | 2 | 3 | 4 |

6. Determination of Human EC-SOD Gene Expression in Mouse Liver Tissue by Real-Time Quantitative Reverse-Transcriptase Polymerase Chain Reaction (RT-PCR)

The mRNA levels of human EC-SOD in mouse liver tissue were determined by real-time quantitative reverse transcription-polymerase chain reaction (RT-PCR) using mouse β-actin as a house-keeping gene control. RNA was extracted from the mouse liver tissue of three independent experiments by TRIzol, and quantitated by absorbance of 260 nm wavelength. cDNA was generated by reverse transcription of DNase I-digested RNA employing ThermoScript RT-PCR Systems. Amplification was performed with Platinum PCR Super Mix and a primer pair of the human EC-SOD gene in ABI Prism 7700 Thermal Cycler (Wege et al., Gastroenterology, 124:432-444 (2003)). The sequences of the primer pair were: the forward primer, 5′-AACTGCCCCGCGTCTTC-3′; the reverse primer: 5′-GCCAAACATTCCCCCAAAG-3′; and a fluorescent probe: 6-carboxyfluorescein-5′-TGTTTCGCATCCACCGCCACC-3′. The concentration for forward primer was 900 nM, reverse primer, 50 nM, both of which were optimized for an annealing temperature of 60° C. Following 40 cycles of amplification in the above condition, semi-log amplification curves were evaluated by comparative quantification (ΔΔC_{T}) (Wege et al., supra). Expression levels were normalized to mouse β-actin house-keeping gene control. The relative gene expression in different groups was calculated based on the average level of the saline control group.

7. Statistical Analysis

Most data were expressed as means±SEM and evaluated by the one-way variance test and Newman-Keuls test for multiple comparisons among groups. Student unpaired t test was used for the comparison between two groups. Wilcoxon Signed Rank test was employed for evaluating quantitative RT-PCR data, followed by q tests for multiple comparisons among groups. A p-value of less than 0.05 was considered as statistically significant.

8. Chemicals and Regents

Eagle's minimum essential medium (MEM), cell culture supplements, and fetal bovine serum were purchased from Invitrogen, Inc. (Gaithersburg, Md.). D-Galactosamine, lipopolysaccharide (endotoxin), hypoxanthine and xanthine oxidase are the products of Sigma Chemical Co. (St. Louis, Mo.). Most other chemicals used were commercially available reagents of analytical grade.

9. Delivery of EC-SOD into Small Size Liver Graft

Small size liver graft transplantation in rats is accompanied with a lower survival rate, poorer graft function, and significant inflammatory reaction in the graft after the transplantation when compared to whole liver transplantation. Liposome-mediated EC-SOD gene delivery is an effective approach to provide protection against ROS-associated donor liver graft damage and to improve graft survival. For this purpose, partial orthotopic liver transplantation (OLT) in small experimental animals, such as rats, is used as a model for assessing the graft function, proliferative potential, and replicability of the transplantation operation.

Successful 20-50% partial OLT in rats has been reported, with a mortality rate inversely corresponding to the graft size. In humans, 40% of standard liver volume is required for living donor liver transplantation in order to achieve sufficient regeneration post transplantation. According to Omura et al. (Transplantation 1996; 62: 292-293), fifty percent of the syngeneic liver grafts regenerated well, achieving a volume nearly equal to that of the recipient's native liver in one to two weeks. Hepatocytes are the major cell type that proliferates in a boosting wave during the first 1-3 days after the transplantation, as indicated by in situ 5-bromo-2′-deoxyuridine (BrdU) or [³H]-thymidine incorporation (Olthoff K M Liver Transplantation 2003; 9: S35-S41; Tanaka H et al., J Surge. Res. 2003; 110: 409-412; Francavilla A, et al., Hepatology 1994; 19:210-216). This is an advantage for liposome-mediated gene delivery, because a high level of transgene expression will be easily achieved in replicating cells with non-viral gene delivery approaches. The present inventors are the first to use a small size liver graft (45% of standard liver volume) in OLT to evaluate the efficacy of ex vivo gene delivery in this model.

Male inbred Lewis rats (RT11) are used as donors and recipients. The donor liver harvest, cuff preparation, and size reduction are performed as described by Omura et al., supra. In brief, under inhalation anesthesia with methoxyflulane, donor livers are harvested with a rapid perfusion of 20 ml of physiological saline or UW solution through the catheter placed in the abdominal aorta. The common bile duct is cannulated with a Teflon stent. The portal vein is dissected, and polyethylene cuffs are inserted into the portal vein and infrahepatic vena cava. The isolated graft is put in a container filled with ice-cold saline or the UW solution for graft reduction. The left lateral lobe, left portion of the median lobe, and two caudate lobes are separately removed with the ligation. The reduced graft is composed of the right portion of the median lobe and right lobe. The small size graft is transplanted into inbred body weight-matched rats following a two-cuff method described by Kamada N et al., Transplantation 1979; 28: 47-50.

In the procedure of OLT in rats, there are three opportunities to infuse lipoplexes containing a transgene, e.g., EC-SOD gene. The first is the quick perfusion through the catheter placed in the abdominal aorta. The purpose of this perfusion is to remove the remaining blood in the liver. The perfusate will be drained out of the graft before the blood circulation is re-established. The second opportunity is when the size reduction is completed, and cuffs are inserted into the portal vein and infrahepatic vena cava, and the reduced graft is placed on ice for the implantation in a recipient animal. At this time point, 2-3 ml of lipoplexes containing approximately 1 mg EC-SOD plasmid DNA is injected via the portal vein. The third is when all vessels are connected and the blood circulation is re-established. In one study, the second and third time points are chosen to infuse lipoplexes for ex vivo gene delivery, where there are three transplantations at each time point plus one group of the small size graft transplantation without EC-SOD gene delivery. In each group, one recipient animal is sacrificed on each of the first three days after the transplantation, and the graft will be removed for the examination of the transgene expression: human EC-SOD mRNA levels by RT-PCR, immunohistochemical staining of EC-SOD in liver tissue, and liver and serum SOD activity. The results are compared with small graft transplantation without EC-SOD gene delivery. In addition, serum ALT and bilirubin are assayed, and liver histology is further examined to determine the efficacy of liposome-mediated EC-SOD gene delivery in improving graft functions in the given time period. These initial studies determine the suitable time point for gene delivery in order to achieve optimal level of protection.

Based on the initial study results, further experiments using a large group of animals are conducted to focus on graft growth and survival rate, as well as long term graft function. There are 5 groups in this experiment: A. Whole liver transplant control; B. 45% small graft transplant control; C. Small size graft transplant plus EC-SOD lipoplex infusion; D. Small size graft transplant plus catalase lipoplex infusion; and E. Small size graft transplant plus control lipoplex infusion. There are 16 recipient animals in each group and therefore at least 160 rats are required for donors and recipients for this experiment. 80 recipient animals are used for a short term observation of liver regeneration as detailed below, and the remaining recipient rats for survival observation. For survival observation, recipient animals have independent records of their recovery and survival after the transplantation, and the total observation duration is three weeks. One recipient rat is sacrificed in the first, second, and third weeks after the transplantation for collection of blood samples and liver tissue. Two recipient rats in each group are maintained for two months when there are enough survived animals. The lowest survival rate in control groups is approximately 50% by three weeks, and there are enough animals available for long term survival observation and for evaluation of transgene expression in the graft during the first three weeks following transplantation.

The liver grafts collected at the first, second, and third weeks or two months post transplantation are weighed and compared to their weight before transplantation as well as to their native liver weight. The liver specimens are separately preserved for different usages, such as formalin fixation for hematoxylin-eosin staining; snap frozen for frozen section (in situ staining of apoptosis), RNA or protein extraction to determine the transgene expression at RNA and protein levels; and for lipid peroxidation parameters, such as glutathione levels, MDA and HO⁻., and ONOO.⁻ by electron spin resonance (ESR). Blood samples are used for liver function tests, SOD activity, EC-SOD content, and neutrophil separation when its volume is adequate. Neutrophils are useful for PMA-initiated chemoluminescence as an indication of O₂⁻. generation. These parameters are used for evaluating graft function and transgene activities in addition to animal survival rate.

One goal of the above-described experiments is to determine whether prior anti-oxidative gene delivery will affect graft growth. Limited literature exists regarding which factors affect the growth of a small size graft after transplantation (Meatani, Transplantation 2003; 75: 97-102; Lee S G et al., J Korean Med Sci 1998; 13:350-354). The crucial factors that may affect the growth of a graft include graft size, surgical procedure, and inflammation associated with oxidative stress in the graft. As discussed in previous sections, the inflammation that occurred in the small size graft is similar to the I/R injury seen in OLT. The small size graft itself serves as the stimulus for the graft to grow. However, little is known as to what are the main driving forces to enhance the proliferation in the small size graft: whether they are cytokines or stress-responding hormones. Hepatocyte growth factor (HGF), epidermal growth factor (EGF), TNF-α, interleukin 6 (IL-6), and TGF-α are cytokines which have been shown to be the most important mediators of liver regeneration after partial hepatectomy (Diehl A M. Liver regeneration. Front Biosci 2002; 7:e301-314), and there are many similarities in liver growth between the remaining liver after partial hepatectomy and small size graft; however, a faster growth rate was observed in the recipients than living donors in humans (Kamel I R, et al., Abdom Imaging 2003; 28: 53-57). Whether these cytokines play the same roles in small size graft growth after transplantation is not clear, since a clinical observation showed HGF and TGF-β1 contributed to regeneration of small size liver graft immediately after transplantation (Ninomiya M, et al., Transplantation International 2003; 16: 814-819); whereas, TGF-β1 usually inhibits hepatocyte proliferation. In addition, cyclin D1 is a cell cycle-specific protein, which binds to cyclin D-dependent kinase (cdk) and forms cyclin D1-cdk4 complexes for the regulation of cells to enter the proliferative cycle phase (Allan A L, et al., J Biol Chem 2001; 276: 27272-27280; Jaumot M, et al., Hepatology 1999; 29: 385-395). Tissue levels of cyclin D1 has been shown to be associated with the peak wave of cell proliferation (Kato A, et al., Biochem Biophys Res Commun 1998; 245: 70-75). Thus, cyclin D1 levels are monitored by Western blot analysis in small size graft during the first three days, in addition to the levels of the above-named cytokines in serum and liver, which is described in the below.

To better study the effects of EC-SOD or/catalase gene delivery on the growth of small size graft, hepatocyte proliferation is monitored by in situ BrdU incorporation (Liu et al., Gene Ther 2003; 10: 180-187) or immnohistochemical staining of proliferative cell nuclear antigen (PCNA) (Tian Y H, et al., Liver Transplantation 2003; 9: 789-795), and cyclin D1 levels are monitored as indicators of liver regeneration in the first three days after transplantation. Graft weight is one direct parameter of graft growth, but may not have a big change at an early stage (e.g., the first three days post-transplant). Serum levels of cytokines named above are monitored by commercially available ELISA kits at 1, 4, 8, 24, 48, and 72 hours post-transplantation (serum samples are collected from rat tail vein without sacrificing animals). Liver specimens are collected at day 1, 2, and 3 post-transplantation (sacrificing two recipient rats each days). Above-named cytokine mRNA levels in liver tissue are determined by real time quantitative RT-PCR. The levels of ROS, such as O₂−., H₂O₂, HO⁻., or ONOO.⁻, in liver graft tissue from animals receiving EC-SOD or catalase gene delivery or control lipolexes are determined using techniques described above on each of the first three days.

An alternative approach for achieving a high level of transgene expression is to perform liposome-mediated gene delivery when the targeting cells are in a proliferative state. Thyroid hormone (T₃) is injected to promote hepatocyte proliferation for the gene deliver in normal mice, which resulted in a huge increase in the expression of reporter genes or functional genes in some previous studies (Liu et al., Gene Ther 2003; 10: 180-187). When a small size liver graft is transplanted in a recipient, the graft undergoes a very rapid regeneration to reach its normal size in one to two weeks (Francavilla A, et al., Hepatology 1994; 19:210-216). This provides an opportunity to perform liposome-mediated gene delivery in a small size graft with an ex vivo approach as described above, or before a graft is harvested from a donor. Thus, one alternative time point for gene delivery in a small size graft is a few hours to one day before a graft is harvested from the donor. This approach avoids the use of thyroid hormone to promote hepatocyte proliferation, although thyroid hormone is included in a cocktail used in cadaveric donors for improving donor organ quality. This particular method of delivering gene prior to organ harvest from donor animals is a new approach for improving graft quality and survival. After the lipoplexes are injected via the portal vein, liver cells take up the complexes via endocytosis into the cytosol. The surgical procedure for the graft harvest, size reduction, and perfusion does not affect endocytosized lipoplexes. After the graft is implanted in a recipient, the regeneration starts a few hours after the circulation is re-established. Significant transgene expression are seen in the graft with this novel approach of gene delivery.

### Results

1. SOD Activity in Culture Medium After Transient or Stable Transfection of an EC-SOD Plasmid in Hep G₂ and Hep 3B Cells

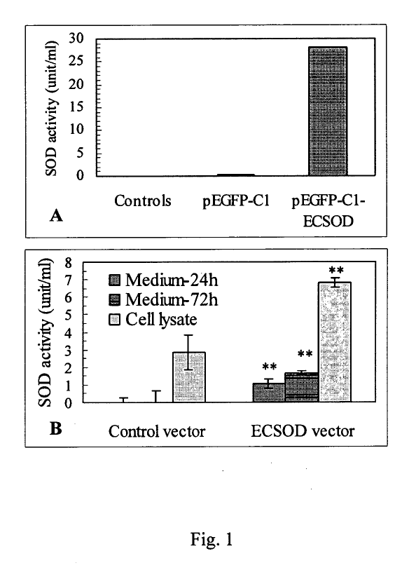

After the verification by restriction enzyme cleavage and sequencing, the new subdloned EC-SOD plasmid, pEGFP-C1-ECSOD, was used to transfect human hepatoma cell lines Hep G₂ and Hep 3B, either transiently or stably. After transient transfection in Hep G₂ cells with pEGFP-C1-ECSOD plasmid DNA, SOD levels in the culture medium were elevated from the background to 28 unit/ml at 72 hours with a medium change to serum-free medium at 24 hours (FIG. 1A). Untransfected cells or cells transfected with the control plasmid, pEGFP-C1, did not show any increase in SOD activity in medium. Hep 3B cells were transfected with either the control plasmid or EC-SOD plasmid by our PCL-Chol liposome formulation (3:1 molar ratio, 5:1 charge ratio), and the transfected cells were selected by the addition of geneticin (G418 at 350 μg/ml). G418 resistant cells were cultured and the medium was changed to serum-free medium. SOD activity in culture medium and cell lysates from EC-SOD plasmid-transfected Hep 3B cells was markedly increased 24 and 72 hours after the medium change compared to the control plasmid-transfected cells (p<0.01, FIG. 1B). The SOD activity measured reflects total activity of all three SOD isoforms in the cell lysates.

2. Protection Against Toxicity of Superoxide Anions and Hydroxyethyl Radicals by EC-SOD Overexpression in Hep G₂ Cells

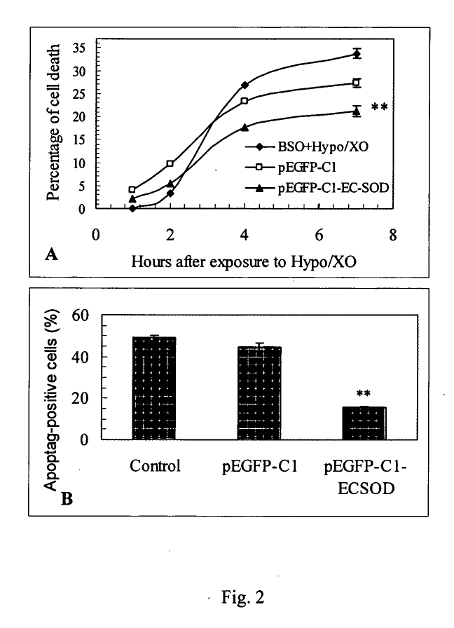

One day after the transfection of Hep G₂ cells with either control plasmid or EC-SOD plasmid, the cells were first exposed to buthionine sulfoximine (BSO) at 0.3 mM for 18 hours to deplete GSH, and subsequently to hypoxanthine (HX, 1.0 mM) and xanthine oxidase (OX, 2 milli-units) for up to 7 hours. Culture medium was sampled for the determination of LDH leakage from the cells. As shown in FIG. 2A, LDH leakage from the cells transfected with pEGFP-C1-ECSOD at a late time point (7 hours after exposure to HX/OX) was significantly lower than untransfected cells or cells transfected with the control plasmid, pEGFP-C1 (p<0.01). EC-SOD plasmid-transfection also markedly diminished the number of Apoptag-positive cells after HX/XO-exposure (FIG. 2B).

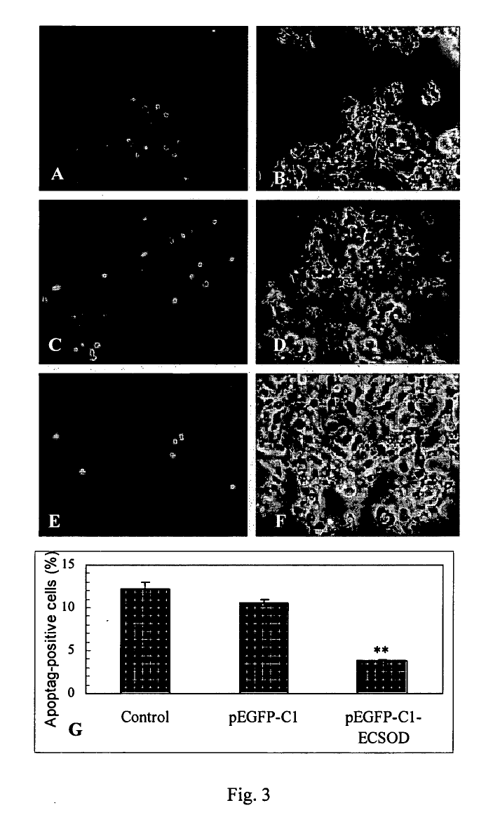

In separate experiments, after Hep G₂ cells were treated with BSO, they were subsequently exposed to an HER-generating system, which consists of H₂O₂ (0.1 mM), ferrous ammonium sulfate (20 μM), and ethanol (200 mM), for 8 hours. An in situ apoptosis detection kit was employed to stain the apoptotic (Apoptag-positive) cells (FIG. 3). It is clear that cells transfected with the EC-SOD plasmid had a much lower percentage of Apoptag-positive cells (FIG. 3E) compared to untransfected controls (FIG. 3A) or to the control plasmid-transfected cells (FIG. 3C). Apoptag-positive cell counts shown in FIG. 3G quantitate the findings.

3. PCL-Chol-Mediated EC-SOD Gene Delivery to Mouse Liver and Protection Against GalN/LPS-Induced Acute Liver Toxicity

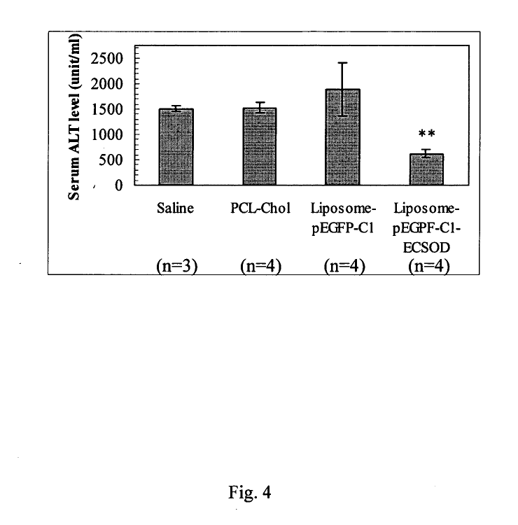

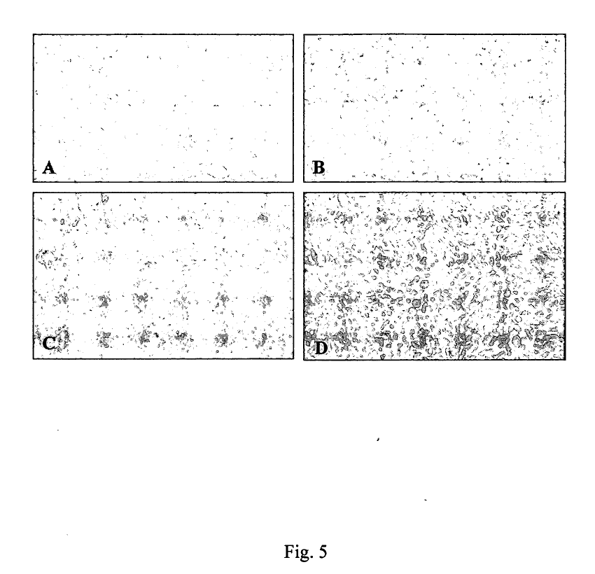

Following a protocol described in Table 1 and the time line, we first injected T₃ subcutaneously (4 mg/kg). Two days after T₃ injection, PCL-Chol liposome or lipoplexes were injected via the portal vein. One day after liposome or lipoplex injection, all animals were challenged intraperitoneally with GalN (500 m/kg) plus LPS (25 μg/kg), then sacrificed one day later. Serum ALT levels in animals receiving liposomes alone or lipoplexes with control plasmid were similar to levels in saline controls (p>0.05). Serum ALT levels in animals receiving EC-SOD lipoplexes (PCL-Chol-pEGFP-C1-ECSOD) were markedly lower than the other three groups (41%, p<0.01) (FIG. 4). Liver histology revealed a similar degree of massive cell death and inflammatory infiltration in GalN/LPS-intoxicated animals plus portal vein injection of saline, liposomes (PCL-Chol) or control lipoplexes (PCL-Chol-pEGFP-C1) (FIGS. 5A, B, and C). Much less cell death was found in the liver of animals receiving EC-SOD lipoplexes (PCL-Chol-pEGFP-C1-ECSOD) (FIG. 5D) before the GalN/LPS challenge, compared to the other three groups. Thus, the liver histology findings are consistent with serum ALT level alterations, and liver injury in mice receiving EC-SOD lipoplexes (PCL-Chol-pEGFP-C1-ECSOD) was markedly attenuated compared to those treated with saline control, PCL-Chol liposomes only, and control lipoplexes (PCL-Chol-pEGFP-C1).

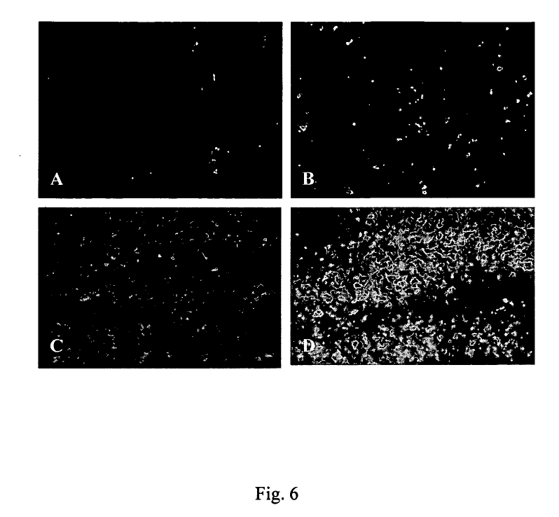

Liver frozen sections were examined for GFP expression. As shown in FIG. 6, intensive GFP expression was found in the liver sections from animals receiving control lipoplexes (PCL-Chol-pEGFP-C1) or EC-SOD lipoplexes (PCL-Chol-pEGFP-C1-ECSOD) (C & D), but not in the sections from saline control mice or from liposome-injected mice (A & B). This figure demonstrates that the approximately 60-70% of liver cells were GFP-positive, indicating that this portion of liver cells were transfected with GFP-ECSOD plasmid and expressed GFP as well as EC-SOD, because the two genes were under the control of the same promoter and in the same reading frame.

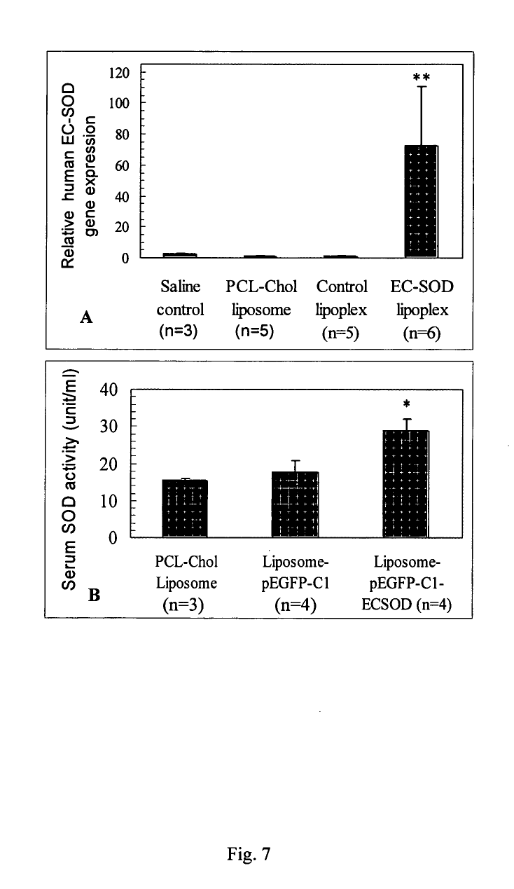

Human EC-SOD gene expression in the livers of mice receiving injections of either PCL-Chol liposomes or control lipoplexes (PCL-Chol-pEGFP-C1) did not significantly differ from the saline control group, as evaluated by real time quantitative RT-PCR, using the mouse β-actin as a house-keeping gene control. Mouse EC-SOD does not cross hybridize with the human gene. The liver human EC-SOD gene expression levels in mice receiving EC-SOD lipoplex (PCL-Chol-pEGFP-C1-ECSOD) injection were approximately fifty five-fold higher than the other three groups (FIG. 7A). Serum-SOD activity was measured in all GalN/LPS-intoxicated mice, and the data showed that total SOD activity in the serum of animals receiving portal vein injection of EC-SOD lipoplexes (PCL-Chol-pEGFP-C1-ECSOD) was higher than in those receiving the injection of liposomes alone or control lipoplexes (p<0.01) (FIG. 7B). The SOD activity measured reflects the three human and mouse isoforms of SOD in serum. Thus, the results indicate that the gene was successfully delivered to the mouse liver with our polycationic liposomes, and that the gene was highly expressed in the mouse liver.

4. GSH Preservation and Reduced Lipidperoxidation by EC-SOD Gene Delivery in GalN/LPS-Intoxicated Mice

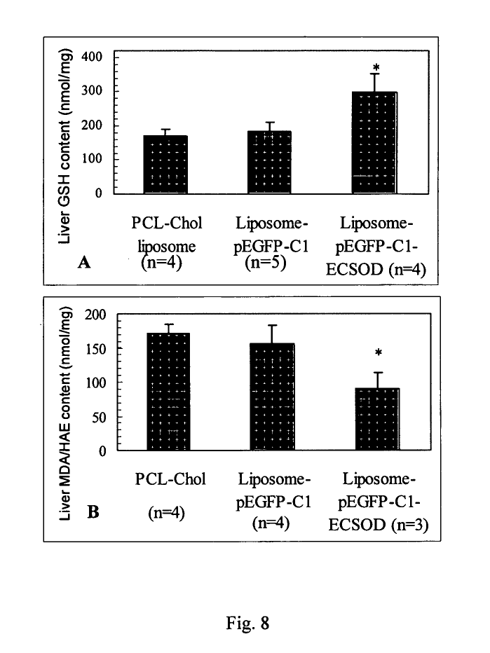

In acute liver injury induced by GalN-sensitized LPS toxicity, enhanced lipid peroxidation was shown by decreased levels of the reduced form of glutathione (GSH), and by elevated MDA and HAE levels in the liver. Animals receiving EC-SOD gene delivery had preserved GSH levels (p<0.05), and reduced MDA/HAE levels (p<0.05) in their livers in comparison with liposome controls and control plasmid-transferred animals (FIGS. 8A, B).

### Discussion

In the present study, we first tested whether the newly subcloned EC-SOD plasmid works in hepatoma cell lines after transfection. Elevated SOD activity was seen in culture medium from Hep G₂ cells transiently transfected and from Hep 3B cells stably transfected with the new recombinant EC-SOD plasmid, pEGFP-C1-ECSOD. Increased SOD levels in culture medium demonstrate the characteristic extracellular location of the enzyme. We then treated control plasmid-transfected or EC-SOD plasmid-transfected Hep G₂ cells initially with BSO, a GSH-depleting agent (Wu et al., Alcohol Clin. Exp. Res., 25:619-628 (2001)), and subsequently with either superoxide anions generated from hypoxanthine (HX) and xanthine oxidase (XO) (Halliwell et al., Free Radicals in Biology and Medicine, 2^{nd} edition, Clarendon Press, Oxford, pp86-187 (1989)), or with hydroxyethyl radicals (HER) by an HER-generating system (Sakurai et al., Free Rad Biol Med, 28:2:273-280 (2000)). The cells transfected with pEGFP-C1-ECSOD showed less LDH leakage and a lower percentage of Apoptag-positive cells compared to those transfected with the control plasmid or untransfected cells. Thus, the results indicate that the overexpression of the EC-SOD makes cells more resistant to superoxide anions (.O₂⁻) or other superoxide anion-derived ROS, such as hydroxyl radicals (HO⁻.) or peroxynitrite anions (ONOO⁻.) (Wu et al., Exp Opin Invest Drugs, 8:585-607 (1999); Jaeschke et al., Toxicol Sci, 65:166-176 (2002)). A similar finding was achieved in Hep G₂ cells when they were treated with HER, which occurs in ethanol metabolism by cytochrome P-450 2E1, and contributes to the pathogenesis of alcohol-associated liver injury (Cederbaum et al., Free Rad Biol Med, 31:1539-1543 (2001)).

In previous studies (Wu et al., Bioconjugate Chem, 12:251-257 (2001); Liu et al., Gene Ther, 10:180-187 (2003)), polycationic liposomes generated from PCL and cholesterol have been reported to be serum-resistant in vitro and interact the least with plasma proteins in the bloodstream when compared to two other commonly used cationic liposome formulations, DOTAP-Chol and DOTAP-DOPE (Templeton et al., Nature Biotechnol, 15:647-652 (1997); Ramesh et al., Mol. Ther., 4:337-350 (2001)). In addition, a non-invasive approach has also been developed to promote hepatocyte proliferation by the injection of thyroid hormone (T₃) for liposome-mediated liver gene delivery. T₃ injection led to a level of DNA synthesis in hepatocytes similar to partial hepatectomy, and markedly enhanced reporter gene expression in the liver when polycationic liposome-plasmid DNA complexes (lipoplexes) were administered either via tail vein or portal vein (Liu et al., Gene Ther., 10:180-187 (2003)). In the present study, the same approach is employed to deliver the EC-SOD gene with the polycationic liposome formulation to the liver via portal vein injection. The markedly enhanced human EC-SOD gene expression in the mouse liver tissue and elevated serum SOD activity in the animals receiving EC-SOD lipoplex (PCL-Chol-pEGFP-C1-ECSOD) injection via the portal vein confirmed that the hepatocytes were transfected, expressed the functional gene, and released the active enzyme into the bloodstream. Both the control plasmid- and EC-SOD plasmid-transfected liver cells showed positive GFP expression at a high rate (FIG. 6), which further confirms the effectiveness of the polycationic liposome-mediated gene transfer to the liver (Liu et al., Gene Ther., 10:180-187 (2003)).

LPS-induced acute liver injury in GalN-sensitized mice is a common model of ROS-associated toxicity, in which enhanced lipid peroxidation is thought to be responsible for the hepatocellular death, via necrosis or apoptosis (Nakama et al., Hepatology, 33:1441-1450 (2001)). Elevated levels of tumor necrosis factor-α (TNF-α) and ROS contribute to the enhanced lipid peroxidation and GSH depletion (Bang et al., J. Pharmacol. Exp. Ther., 305:31-39 (2003); Garcia-Ruiz et al., J. Clin. Invest., 111:197-208 (2003); Dai et al., Gene Ther., 10:550-558 (2003)). Findings in this study, including elevated serum ALT, massive cell death and inflammatory infiltration in the liver sections, as well as decreased GSH levels and elevated MDA/HAE in the liver tissue, demonstrate the feasibility of the model as a means of determining the therapeutic efficacy of liposome-mediated EC-SOD gene delivery. Consequently, a marked decrease in serum ALT levels, improved liver histology, preserved GSH content, and decreased MDA/HAE content in the liver tissue from animals receiving the portal vein injection of EC-SOD lipoplexes compared to those receiving saline, liposomes alone, or liposome-control plasmid documented the efficacy of the EC-SOD gene delivery. The protection can be attributed to the enhanced scavenging activity of EC-SOD in the liver cells and in the interstitial space. These results are consistent with a recent report that topical transfer of EC-SOD gene ameliorated antigen-induced arthritis in rats (Dai et al., Gene Ther., 10:550-558 (2003)), and that adenoviral vector-mediated EC-SOD gene delivery attenuated acetaminophen liver toxicity (Laukkanen et al., J. Gene Med., 3:321-325 (2001)). As was demonstrated in our previous studies, and in this study by each of increased ALT levels receiving PCL-Chol liposomes alone, our liposomal formulation is non-toxic in vivo. Slightly increased, but not statistically significant, serum ALT levels in the PCL-Chol-pEFGP-C1 group suggests a possible toxic effect of plamsid DNA due to the “CpG” motif from the bacterial genomic sequence during the plasmid transformation (Loisel et al., Hum. Gene Ther., 12:685-606 (2001)). This effect can be eliminated by further purification of plasmid DNA (Yew et al., Mol Ther., 5:731-738 (2002)). Thus, our results suggest that liposome-mediated delivery of a functional gene is at least as good as adenoviral delivery (Laukkanen et al., J. Gene. Med., 3:321-325 (2001)), if not better. The reason for this is because high titers of adenoviruses (titers high enough to be therapeutically significant) may cause liver injury through a direct toxic effect or through immune-mediated damage (Liu et al., Gene Ther., 10:935-940 (2003)).

The fact that liposomes are non-immunogenic enhances their roles as a non-viral vector for gene delivery. Because of that fact, they can be administered repeatedly when a therapy regimen requires a high level of gene expression for an extended period of time. When targeting approaches are desirable, liposomes can be employed to selectively deliver therapeutic genes to a preferential organ or cell type (Wu et al., Front Biosci., 7:d717-725 (2002); Kren et al., Proc. Natl. Acad. Sci. USA, 96:10349-10354 (1999); Kawakami et al., Pharm Res, 17:306-313 (2000)). Our polycationic liposomes display the least reactivity with serum proteins and a better gene transfer efficacy when compared with other commercially available formulations (Liu et al., Gene Ther., 10:180-187 (2003)). One disadvantage associated with non-viral gene delivery approaches and retroviral vectors is that a high level of transgene expression can be achieved only when targeted cells are in a proliferative state (Liu et al., Gene Ther., 10:180-187 (2003)). We employed a non-invasive approach to promote hepatocyte proliferation by the injection of thyroid hormone before administering the lipoplexes. This approach resulted in a level of hepatocyte proliferation similar to partial hepatectomy (Liu et al., Gene Ther., 10:180-187 (2003)), and a high level of the transgene expression was detected in the mouse liver, leading to functional consequence, protection against oxidative injury.

In summary, the findings in the present study demonstrate that the overexpression of extracellular superoxide dismutase protects against either superoxide anion- or hydroxyethyl radical-induced necrosis/apoptosis in Hep G₂ cells, and that polycationic liposome-mediated EC-SOD gene delivery markedly attenuated GalN/LPS-induced acute liver injury in mice. This is the first report demonstrating that polycationic liposome-mediated EC-SOD gene delivery to the liver represents a potential therapy for reactive oxygen species-associated liver injury.

All patents, patent applications, and other publications cited in this application are incorporated by reference in the entirety.

TABLE 2
SEQ ID NO:1 Human EC-SOD cDNA sequence (GenBank Accession No. NM_003102, ORF shaded)

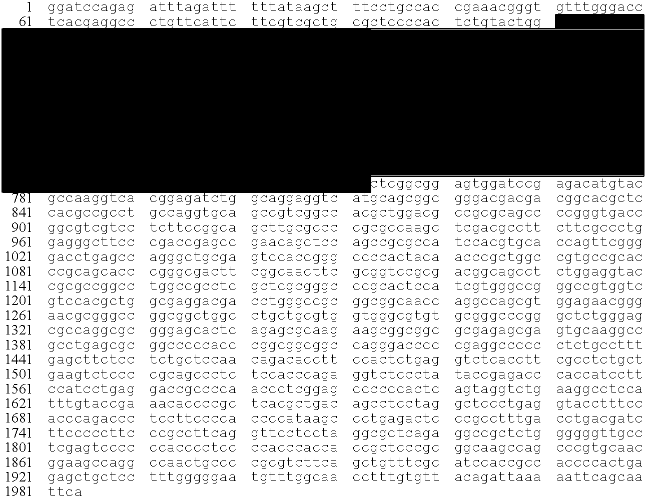

SEQ ID NO:2 Human EC-SOD coding sequence (nucleotides 111-752 of GenBank Accession No. NM_003102)

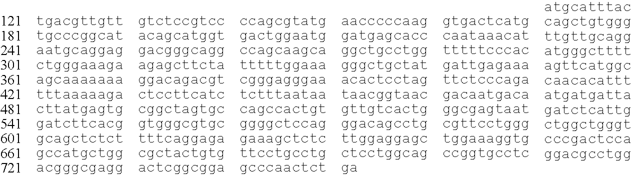

SEQ ID NO:3 Human EC-SOD amino acid sequence, GenBank Accession No. AAA66000

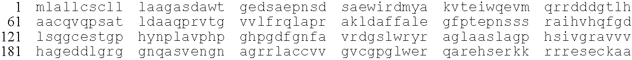

What is claimed is:

1. A method for preventing oxidative injury to a cell, the method comprising the step of contacting the cell with a composition, which comprises a lipid and a recombinant nucleic acid that comprises a polynucleotide encoding an extracellular superoxide dismutase (EC-SOD), whereby the EC-SOD is expressed by the cell.

2. The method of claim 1, wherein the nucleic acid is not in a viral vector.

3. The method of claim 1, wherein the lipid is a polycationic lipid.

4. The method of claim 1, wherein the lipid is polycationic acrylamide lipid (polyAL).

5. The method of claim 1, wherein the lipid is conjugated to asialofetuin (AF) or galactose.

6. The method of claim 3, wherein the composition further comprises cholesterol.

7. The method of claim 1, wherein the cell is a part of an organ.

8. The method of claim 7, wherein the organ is a liver.

9. The method of claim 7, wherein the organ is a section of a liver.

10. The method of claim 1, wherein the contacting step is performed in vivo.

11. The method of claim 10, wherein the composition is delivered to the cell by intravenous injection.

12. The method of claim 7, wherein the contacting step is performed in vitro and the method further comprises the step of transplanting the organ into an animal.

13. The method of claim 12, wherein the animal is a human.

14. The method of claim 1, wherein the polynucleotide sequence encodes the amino acid sequence of SEQ ID NO:3.

15. The method of claim 14, wherein the polynucleotide sequence is SEQ ID NO:2.

16. The method of claim 1, wherein the nucleic acid further comprises a promoter, which directs the expression of the polynucleotide sequence.

17. The method of claim 1, wherein the promoter is the cytomegalovirus (CMV) promoter.

18. The method of claim 1, wherein the promoter is a tissue-specific promoter.

19. The method of claim 18, wherein the tissue-specific promoter is a liver-specific promoter.

20. A composition comprising a lipid and a recombinant nucleic acid, wherein the recombinant nucleic acid comprises a polynucleotide sequence encoding an extracellular superoxide dismutase (EC-SOD).

21. The composition of claim 20, wherein the recombinant nucleic acid is not in a viral vector.

22. The composition of claim 20, wherein the lipid is a polycationic lipid.

23. The composition of claim 22, wherein the lipid is a polycationic acrylamide lipid (polyAL).

24. The composition of claim 22, wherein the lipid is conjugated to asialofetuin (AF) or galactose.

25. The composition of claim 22, further comprising cholesterol.

26. The composition of claim 20, wherein the polynucleotide sequence encodes the amino acid sequence of SEQ ID NO:3.

27. The composition of claim 26, wherein the polynucleotide sequence is SEQ ID NO:2.

28. The composition of claim 20, wherein the nucleic acid further comprises a promoter, which directs the expression of the polynucleotide sequence.

29. The composition of claim 20, wherein the promoter is the cytomegalovirus (CMV) promoter.

30. The composition of claim 20, wherein the promoter is a tissue-specific promoter.

31. The composition of claim 30, wherein the tissue-specific promoter is a liver-specific promoter.

32. An isolated organ that has been contacted with a composition, which comprises a lipid and a recombinant nucleic acid comprising a polynucleotide sequence encoding an extracellular superoxide dismutase (EC-SOD), whereby at least some cells of the organ express the EC-SOD.

33. The organ of claim 32, wherein the polynucleotide sequence encodes the amino acid sequence of SEQ ID NO:3.

34. The organ of claim 32, wherein the polynucleotide sequence is SEQ ID NO:2.

35. The organ of claim 32, wherein the nucleic acid further comprises a promoter, which directs the expression of the polynucleotide sequence.

36. The organ of claim 32, wherein the promoter is the cytomegalovirus (CMV) promoter.

37. The organ of claim 32, wherein the promoter is a tissue-specific promoter.

38. The organ of claim 37, wherein the tissue-specific promoter is a liver-specific promoter.

39. The organ of claim 32, which is a liver.

40. The organ of claim 32, which is a section of a liver.


SEQUENCE LISTING (6 sequences)

SEQ ID NO: 1 - DNA, 1984 bp, Homo sapiens
  human extracellular superoxide dismutase (EC-SOD, SOD-3) cDNA
ggatccagagatttagattttttataagctttcctgccaccgaaacgggtgtttgggacctcacgaggccctgttcattcttcgtcgctgcgctccccactctgtactggatgcatttactgacgttgttgtctccgtccccagagtatgaacccccaaggtgactcatgcagctgtgggtgcccggcatacagcatggtgactggaatggatgagcacccaataaacatttgttgcaggaatgcaggaggacgggcaggccagcaagcaggctgcctggtttttcccacatgggcttttctgggaaagaagagcttctatttttggaaagggctgctatgattgagaaaagttcatggcagcaaaaaaaggacagacgtcgggagggaaacactcctagttctcccagacaacacattttttaaaaagactccttcatctctttaataataacggtaacgacaatgacaatgatgattacttatgagtgcggctagtgccagccactgtgttgtcactgggcgagtaatgatctcattggatcttcacggtgggcgtgcggggctccagggacagcctgcgttcctgggctggctgggtgcagctctcttttcaggagagaaagctctcttggaggagctggaaaggtgcccgactccagccatgctggcgctactgtgttcctgcctgctcctggcagccggtgcctcggacgcctggacgggcgaggactcggcggagcccaactctgactcggcggagtggatccgagacatgtacgccaaggtcacggagatctggcaggaggtcatgcagcggcgggacgacgacggcacgctccacgccgcctgccaggtgcagccgtcggccacgctggacgccgcgcagccccgggtgaccggcgtcgtcctcttccggcagcttgcgccccgcgccaagctcgacgccttcttcgccctggagggcttcccgaccgagccgaacagctccagccgcgccatccacgtgcaccagttcggggacctgagccagggctgcgagtccaccgggccccactacaacccgctggccgtgccgcacccgcagcacccgggcgacttcggcaacttcgcggtccgcgacggcagcctctggaggtaccgcgccggcctggccgcctcgctcgcgggcccgcactccatcgtgggccgggccgtggtcgtccacgctggcgaggacgacctgggccgcggcggcaaccaggccagcgtggagaacgggaacgcgggccggcggctggcctgctgcgtggtgggcgtgtgcgggcccgggctctgggagcgccaggcgcgggagcactcagagcgcaagaagcggcggcgcgagagcgagtgcaaggccgcctgagcgcggcccccacccggcggcggccagggacccccgaggcccccctctgcctttgagcttctcctctgctccaacagacaccttccactctgaggtctcaccttcgcctctgctgaagtctccccgcagccctctccacccagaggtctccctataccgagacccaccatccttccatcctgaggaccgccccaaccctcggagccccccactcagtaggtctgaaggcctccatttgtaccgaaacaccccgctcacgctgacagcctcctaggctccctgaggtacctttccacccagaccctccttccccaccccataagccctgagactcccgcctttgacctgacgatcttcccccttcccgccttcaggttcctcctaggcgctcagaggccgctctggggggttgcctcgagtccccccacccctccccacccaccaccgctcccgcggcaagccagcccgtgcaacggaagccaggccaactgccccgcgtcttcagctgtttcgcatccaccgccaccccactgagagctgctcctttgggggaatgtttggcaacctttgtgttacagattaaaaattcagcaattca

SEQ ID NO: 2 - DNA, 642 bp, Homo sapiens
  human extracellular superoxide dismutase (EC-SOD, SOD-3) coding sequence
atgcatttactgacgttgttgtctccgtccccagagtatgaacccccaaggtgactcatgcagctgtgggtgcccggcatacagcatggtgactggaatggatgagcacccaataaacatttgttgcaggaatgcaggaggacgggcaggccagcaagcaggctgcctggtttttcccacatgggcttttctgggaaagaagagcttctatttttggaaagggctgctatgattgagaaaagttcatggcagcaaaaaaaggacagacgtcgggagggaaacactcctagttctcccagacaacacattttttaaaaagactccttcatctctttaataataacggtaacgacaatgacaatgatgattacttatgagtgcggctagtgccagccactgtgttgtcactgggcgagtaatgatctcattggatcttcacggtgggcgtgcggggctccagggacagcctgcgttcctgggctggctgggtgcagctctcttttcaggagagaaagctctcttggaggagctggaaaggtgcccgactccagccatgctggcgctactgtgttcctgcctgctcctggcagccggtgcctcggacgcctggacgggcgaggactcggcggagcccaactctga

SEQ ID NO: 3 - protein, 240 aa, Homo sapiens
  human extracellular superoxide dismutase (EC-SOD, SOD-3)
MLALLCSCLLLAAGASDAWTGEDSAEPNSDSAEWIRDMYAKVTEIWQEVMQRRDDDGTLHAACQVQPSATLDAAQPRVTGVVLFRQLAPRAKLDAFFALEGFPTEPNSSSRAIHVHQFGDLSQGCESTGPHYNPLAVPHPQHPGDFGNFAVRDGSLWRYRAGLAASLAGPHSIVGRAVVVHAGEDDLGRGGNQASVENGNAGRRLACCVVGVCGPGLWERQAREHSERKKRRRESECKAA

SEQ ID NO: 4 - DNA, 17 bp, Artificial Sequence
  Description of Artificial Sequencereal-time quantitative reverse transcription polymerase chain reaction (RT-PCR) forward primer
aactgccccgcgtcttc

SEQ ID NO: 5 - DNA, 19 bp, Artificial Sequence
  Description of Artificial Sequencereal-time quantitative reverse transcription polymerase chain reaction (RT-PCR) reverse primer
gccaaacattcccccaaag

SEQ ID NO: 6 - DNA, 21 bp, Artificial Sequence
  Description of Artificial Sequencefluorescent probe
ngtttcgcatccaccgccacc